(19) United States
(12) Patent Application Publication
(10) Pub. No.: US 2014/0288577 A1
(43) Pub. Date: Sep. 25, 2014

(54) Electrode Assembly for an Active Implantable Medical Device

(71) Applicant: Saluda Medical Pty Limited, Artarmon, NSW (AU)

(72) Inventors: David Robinson, Eveleigh (AU); Mark Fretz, Bueren (CH); David Thomas, Eveleigh (AU); John Louis Parker, Eveleigh (AU)

(73) Assignee: SALUDA MEDICAL PTY LIMITED, Artarmon, NSW (AU)

(21) Appl. No.: 14/359,777

(22) PCT Filed: Nov. 23, 2012
(86) PCT No.: PCT/AU2012/001441

§ 371 Date: May 21, 2014

(30) Foreign Application Priority Data
Nov. 24, 2011 (AU) 2011904903

(51) Int. Cl. A61N 1/05

(52) U.S. Cl. CPC A61N 1/0553
USPC 606/129; 607/116; 29/876

(57) ABSTRACT

An electrode assembly for an active implantable medical device can be delivered by catheter but expands to become a paddle electrode once implanted. The electrode assembly comprises a support member carrying wires for electrically connecting a control unit to electrodes of the electrode assembly. At least one, and usually two resilient deformable paddle wings are mounted to the support member. The paddle wings can be furled close to the support member under a deformation force to permit implantation via an introducer. The paddle wings resiliently unfurl away from the support member upon release of the deformation force. The paddle wings bear rows and columns of electrodes, and the electrode assembly as a whole has sufficient longitudinal rigidity for implantation via an introducer.

### CROSS-REFERENCE TO RELATED APPLICATIONS

This application claims the benefit of Australian Provisional Patent Application No. AU2011904903 filed 24 Nov. 2011, the content of which is incorporated herein by reference.

### TECHNICAL FIELD

The present invention relates generally to active implantable medical devices (AIMDs), and more particularly, to a paddle electrode for an AIMD made using textile techniques.

### BACKGROUND OF THE INVENTION

Medical devices having one or more active implantable components, generally referred to herein as active implantable medical devices (AIMDs), have provided a wide range of therapeutic benefits to patients over recent decades. AIMDs often include an implantable, hermetically sealed electronics module, and a device that interfaces with a patient's tissue, sometimes referred to as a tissue interface. The tissue interface may include, for example, one or more instruments, apparatus, sensors or other functional components that are permanently or temporarily implanted in a patient. The tissue interface is used to, for example, diagnose, monitor, and/or treat a disease or injury, or to modify a patient's anatomy or physiological process.

In particular applications, an AIMD tissue interface includes one or more conductive electrical contacts, referred to as electrodes, which deliver electrical stimulation signals to, or receive electrical signals from, a patient's tissue. The electrodes are typically disposed in a biocompatible electrically non-conductive member, and are electrically connected to an electronics module. The electrodes and the nonconductive member are collectively referred to herein as an electrode assembly.

An AIMD uses electrical power to perform its intended function. An AIMD may sense electrical signals in the body and/or deliver electrical charge into the body, generally for a therapeutic purpose. This kind of device is shown in FIG. 1. In such devices a means is required for electrically connecting the sensing and/or charge delivery electronics to the appropriate body tissue. Typical devices for doing this include catheter (or percutaneous) electrodes (FIG. 2 (A)) paddle electrodes (FIG. 2 (B)) and cuff electrodes (FIG. 2 (C)). Different electrode types have their own area of application, and specific methods of surgical implantation.

In the case of spinal cord stimulators (SCS), for example, a catheter electrode assembly may be introduced into the epidural space percutaneously. FIG. 3 shows the percutaneous insertion of a catheter-style electrode into the epidural space of the spine. It can be seen that a suitable introducer, for example a suitable gauge Tuohy needle, is introduced into the epidural space between the vertebrae and then the catheter electrode is fed through the introducer into the epidural space. The introducer is then withdrawn by sliding it back, over the proximal end of the catheter electrode. This is a minimally invasive procedure, which can be performed by a relatively large number of surgeons. However, as the size of the introducer is limited by the geometry of the spine, this procedure is only suitable for electrodes that can be fed through a suitable introducer. For this reason a catheter electrode assembly is narrow, and only has a single row of electrodes. Accurately positioning a catheter electrode assembly during implantation is important as very small lateral deviations off the dorsal column can significantly affect device performance.

In contrast FIG. 4 shows the procedure for the placement of the larger paddle electrode. In this procedure part of the bony spine (lamina) is removed in a procedure known as a laminectomy. This creates an entry point at least large enough for the paddle electrode to be inserted. Often the laminectomyis larger than this, giving the surgeon the possibility of visualizing the dura mater itself to aid with placement. It will be understood that this procedure gives the implanting surgeon much more flexibility in the implanting procedure at the expense of being much more invasive. The more complex laminectomy procedure typically limits this procedure to neurosurgeons. A paddle electrode assembly, which is broader and comprises two or three or more rows of electrodes, can permit the use of electrode row selection to overcome lateral positional errors arising during surgery or resulting from post-surgical device migration.

While the ease of surgical implantation is a significant factor, different electrode types also have different efficacy in particular therapeutic applications once implanted. For example, it has been reported that paddle electrodes may provide significantly more effective long-term treatment for chronic pain in the lower back and lower extremities. However, despite these results, percutaneously introduced catheter electrodes continue to be a popular style of electrode used in SCS therapies due to the lower invasiveness of that approach.

Implantable medical devices such as AIMDs may make use of textile techniques for part or all of their fabrication. Such methods include knitting (such as warp knitting or weft knitting) and braiding. For example US Patent Application Publication No. 2010/0070008 A1 teaches a method of fabricating a catheter-style electrode assembly using textile techniques.

There are a range of distinct methods for forming textiles. Weaving produces woven fabrics which have two or more thread systems, the thread systems being at an angle (often perpendicular) to each other and referred to as the warp threads and the weft threads. In a weaving loom alternate warp threads are raised or lowered by shafts, a weft thread is inserted between and laterally to the warp threads by a reed or the like, and then the warp threads are moved vertically to the opposite lowered or raised position by the respective shafts and the process repeats.

Braiding is another technique of forming textiles, and involves three or more (often 16, 32 or more) threads which are braided into a fabric in which the threads cross each other diagonally relative to the selvedges to form a braid having even fabric density and a closed fabric appearance.

Knitting is yet another technique of forming textiles. Knits are fabrics which are made of one or more threads, or one or more thread systems, by stitch formation. Stitches are formed from intertwined stitch loops whereby a single continuous length of yarn forms a row of stitch loops, each loop linked with respective loops in the adjacent rows to form a stitch wale and thereby form a two dimensional fabric comprising stitch rows and stitch wales. In plain and purl stitches, a single stitch loop comprises a head, two thighs and two feet, and has only two crossing points at the feet. Once a plain or purl stitch is formed from adjacent interlinked stitch loops, the stitch has two top crossing points at the head and two bottom crossing points at the feet. One distinction of knitting compared to weaving and braiding is that in knitting it is possible to knit a fabric with a single yarn or filament. In implantable medical devices, this difference can provide a key advantage. There are less components and less connections required, thereby reducing sources of possible defects, which is a major setback for any implanted device in terms of cost, convenience and most important, risk to the patient. Knitting techniques include warp knitting and weft knitting. Knitted fabrics can have significantly different characteristics, such as stretch, as compared to woven or braided fabrics.

These methods of forming textiles conventionally produce a two-dimensional fabric, being a fabric in which the path or position of a given thread can be defined with only two coordinates. However, most of these methods of forming textiles can also be configured to form a three dimensional fabric, in which a third coordinate is required to define the path or position of each thread.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY OF THE INVENTION

According to a first aspect the present invention provides an electrode assembly for an active implantable medical device, the electrode assembly comprising:

a support member carrying wires for electrically connecting a control unit to electrodes of the electrode assembly; and

at least one resilient deformable paddle wing mounted to the support member, the paddle wing being configured to be furled close to the support member under a deformation force to permit implantation via an introducer, and the paddle wing being configured to resiliently unfurl away from the support member upon release of the deformation force, the paddle wing bearing at least one electrode.

According to a second aspect the present invention provides a method of constructing an electrode assembly for an active implantable medical device, the method comprising:

forming a support member carrying wires for electrically connecting a control unit to electrodes of the electrode assembly; and

forming at least one resilient deformable paddle wing mounted to the support member, the paddle wing being configured to be furled close to the support member under a deformation force to permit implantation via an introducer, and the paddle wing being configured to resiliently unfurl away from the support member upon release of the deformation force, the paddle wing bearing at least one electrode.

According to a third aspect the present invention provides a method of implanting an electrode assembly for an active implantable medical device, the method comprising:

furling one or more resilient paddle wings of the electrode assembly close to a support member of the electrode assembly;

positioning the furled electrode assembly within an introducer;

delivering an outlet of the introducer to a site of desired implantation; and

ejecting the electrode assembly from the outlet while withdrawing the introducer, to thereby position the electrode assembly at the site of desired implantation and to permit the one or more paddle wings to resiliently unfurl.

Preferred embodiments of the present invention thus provide an electrode assembly which can be implanted via introducer when furled but which is a paddle electrode assembly having greater lateral dimension than the introducer internal diameter when unfurled.

Preferred embodiments of the invention comprise first and second paddle wings, the paddle wings configured to extend in substantially opposed directions from the support member when unfurled.

When viewed along an axis of the support member, the first paddle wing may be configured to be furled clockwise around the support member while the second paddle wing may be configured to be furled anti-clockwise around the support member. To maximise a lateral dimension of the paddle wings when unfurled, the attached edge of the first paddle wing is preferably mounted to the support member proximal to the attached edge of the second paddle wing, while a lateral edge of the first paddle wing when furled is proximal to a lateral edge of the second paddle wing when furled.

Alternatively, when viewed along an axis of the support member, the first and second paddle wings may both be configured to be furled in the same direction (whether clockwise or anti-clockwise) around the support member.

In some embodiments of the invention, the paddle wings may be resilient in a manner such that when unfurled the paddle wings seek to return to a planar position in which the paddle wings reside in a single nominal plane. In such embodiments the plane of the paddle wings may for example contain a nominal axis of the cylindrical supporting member. Alternatively the plane of the paddle wings may be tangential to a cross-sectional profile of the supporting member, such as by being tangential to a cross-sectional circumference of a cylindrical supporting member, or the plane of the paddle wings may be otherwise disposed relative to the support member.

In alternative embodiments the paddle wings may be configured to be resilient in a manner such that when unfurled the wings seek to curve away from the cylindrical supporting member. Such embodiments may for example be advantageous in that the paddle wings when unfurled conform more closely to the curved surface of the dorsal column and provide greater stimulation and measurement coverage of the region of the dorsal column that is of interest. Wider electrode coverage may be of advantage in finding optimum stimulation or measurement sites. Closer conformance to the curved surface of the dorsal column may be beneficial in reducing movement of the electrode as posture changes.

The support member and resilient paddle wings of the electrode assembly may be formed of a resilient substrate of sheet material. In such embodiments electrodes may be formed upon the substrate of sheet material as a printed circuit. Alternatively electrodes may be stitched or embroidered or otherwise formed upon the substrate of sheet material in accordance with the teachings of U.S. Utility patent application Ser. No. 12/549,831 (published as US 2010/0262214), which is hereby incorporated by reference herein.

Alternatively, in some embodiments of the present invention the support member and resilient paddle wings of the electrode assembly may be formed as a knitted fabric electrode assembly, for example in accordance with the teachings of U.S. Utility patent application Ser. No. 12/549,899 (published as US 2010/0070008), hereby incorporated by reference herein.

In preferred embodiments, the knitted fabric electrode assembly is formed by knitting of a single composite yarn, the composite yarn comprising a non-conductive filament having at least one conductive portion. The or each conductive portion of the composite yarn may comprise a conductive filament wound helically around a section of the non-conductive filament. For example, the spacing between adjacent coils of the helically wound conductive filament is preferably of the order of or less than the diameter of the non-conductive filament, whereby the helix offers strain relief so that the conductive filament is not subject to strains put upon the assembly as a whole. The ratio of the diameter of the non-conductive filament to the diameter of the conductive filament should exceed the minimum bend radius for the conductive filament. In many practical cases this ratio will be between 4 and 6. The knitting is preferably warp knitting. Three dimensional knitting, such as is effected by a double layer knitting machine, may be used to form the electrode assembly.

In embodiments where the electrode assembly is formed of a knitted fabric, the knitting parameters are preferably selected in order to provide the paddle wings with the desired amount of resilience to permit the wings to be furled within the introducer and to unfurl when released during implantation against the resistance of the surrounding tissue or fluid. Such knitting parameters may include the filament resilience, filament diameter, stitch selection, yarn tension during knitting, and the like. In the preferred embodiment the knitted structure would be produced on a fine gauge (e.g. 16 needles per inch) v-bed knitting machine with a mono-filament yarn made from a suitable biocompatible polymer such as PEEK 50 micron filament. A suitable knitting pattern is represented in FIGS. 11 (b) and (c). Such a structure will be substantially flat as produced from the machine, with the resilience of the wings being provided by the torsional resilience of the mono-filament used in the structure. The insertion of a suitable lumen tube (e.g. a 500 micron outer diameter polyethylene tube) into the central section of the structure (region 1102 in FIG. 11 (b)) will create the supporting member described above. In another embodiment of the invention the wing resilience may be effected by first knitting the assembly from a softer, multi-filament yarn, and then impregnating the knitted assembly with a suitable polymer (e.g. silicone). The latter approach has the advantage that polymers with different durometer rating could be used in different parts of the structure, albeit at the expense of a more complex fabrication process.

The introducer may be a hypodermic needle such as a Tuohy needle or a broader device such as an Epiducer™ from St Jude Medical. The implantation may be percutaneous. The implantation site may be the epidural space, with the introducer entering via the ligamentum flavum.

In some embodiments the electrode assembly has a longitudinal rigidity sufficient for implantation via an introducer. In alternative embodiments the electrode assembly may have reduced longitudinal rigidity, with implantation being effected by insertion of a stylet into the hollow support member during implantation, so that once the electrode assembly is implanted and the stylet and introducer withdrawn the assembly is of reduced longitudinal rigidity.

The electrode assembly may be configured to be steerable, in accordance with the teachings of U.S. Utility patent application Ser. No. 12/549,801 (US 2010/0069835), hereby incorporated by reference herein.

According to a fourth aspect the present invention provides a biocompatible composite filament comprising:

an insulated conductive cable having a conductor and insulating sheath which are both biocompatible;

an exposed length of the conductor, having a free end and a bound end, the free end of the exposed conductor being wound around the outer surface of the filament to form an electrode element; and

a second portion of filament joined to the insulated conductive cable proximal to the bound end of the exposed conductor and in coaxial alignment with the insulated conductive cable.

According to a fifth aspect the present invention provides a method of forming a biocompatible composite filament, the method comprising:

providing an insulated conductive cable having a conductor and insulating sheath which are both biocompatible;

stripping a portion of the insulating sheath, to expose a length of the conductor having a free end and a bound end;

winding the free end of the exposed conductor around the outer surface of the filament to form an electrode element; and

joining a second portion of filament to the insulated conductive cable proximal to the bound end of the exposed conductor and in coaxial alignment with the insulated conductive cable.

The free end of the exposed conductor may be wound around the insulating sheath of the conductive cable, or may be wound around the second portion of filament.

The second portion of filament may be wholly non-conductive, or may have an insulated conductor. The second portion of filament may be formed from the same length of insulated conductive cable by cutting the insulated conductive cable prior to the stripping step. A conductor of the second filament may be electrically connected to the bound end of the exposed conductor to effect an electrical connection past the join, for example to enable multiple electrode contacts to be driven by a single signal. Alternatively, the join may be insulated to prevent electrical contact across the join, for example to maximise power output by the formed electrode.

The second portion of filament and the insulated sheath preferably comprise thermosoftening materials, and are joined by heat fusing. Other joining or bonding methods such as gluing and knotting are also possible.

In preferred embodiments, a plurality of electrodes are formed on the biocompatible composite filament in accordance with the method of the fifth aspect, the electrodes being formed at locations along the composite filament which correspond to desired electrode locations in a fabric electrode assembly intended to be formed from the composite filament. Such embodiments may be particularly advantageous for construction of braided or warp knitted fabric electrode assemblies.

The fourth and fifth aspects of the invention thus provide for a composite filament having one or more exposed electrode elements at precisely defined positions along the filament. This in turn permits the composite filament to be stored on a reel or bobbin and used in a knitting or braiding process without requiring electrode formation to occur during the fabric formation process. Thus, some embodiments of the first through third aspects of the invention may be formed using a composite filament in accordance with the fourth aspect of the invention. In particular, an electrode assembly formed in accordance with the second and fifth aspects may comprise a braided fabric electrode assembly. In such embodiments the use of the composite filament produced by the fourth aspect is important in enabling formation of a braided fabric from a composite filament having the conductive portions (to serve as electrodes) formed into the fabric at predefined locations but without requiring electrode formation during the braiding process.

### BRIEF DESCRIPTION OF THE DRAWINGS

An example of the invention will now be described with reference to the accompanying drawings, in which:

FIG. 1 generally illustrates an active implantable medical device having a catheter electrode assembly;

FIGS. 2a-2c illustrate catheter, paddle and cuff electrode assemblies, respectively;

FIG. 3 illustrates the percutaneous insertion of the catheter-style electrode of FIG. 2a into the epidural space of the spine;

FIG. 4 illustrates the laminectomy procedure for the placement of the larger paddle electrode of FIG. 2b into the epidural space of the spine;

FIG. 5 is a system schematic of an active implantable medical device having a knitted paddle electrode assembly in accordance with one embodiment of the invention;

FIG. 6 illustrates a paddle electrode assembly having resiliently deformable paddle wings in accordance with one embodiment of the invention;

FIG. 7 illustrates a paddle electrode assembly having resiliently deformable paddle wings in accordance with another embodiment of the invention;

FIG. 8 illustrates warp knitting and salient features of the warp knitted fabric;

FIG. 9 illustrates an embodiment of the present invention in which an electrode assembly is formed by alternately knitting with conductive and non-conductive filaments;

FIG. 10a illustrates a composite conductive filament formed by winding a section of a conductive filament around a section of a non-conductive filament; and FIG. 10b illustrates an embodiment of the present invention in which an electrode assembly is formed from the composite filament of FIG. 10a by knitting;

FIG. 11a illustrates a process of knitting a three dimensional knitted fabric paddle electrode assembly using V-bed weft knitting; FIG. 11b illustrates a suitable stitch pattern in accordance with one embodiment of the invention; FIG. 11c illustrates a suitable stitch pattern in accordance with another embodiment of the invention; FIG. 11d illustrates a knitted fabric electrode assembly constructed from yarn; and FIG. 11e is a detail view of the assembly of FIG. 11d;

FIG. 12 is a high level flowchart illustrating a method for manufacturing a knitted paddle electrode assembly in accordance with some embodiments of the present invention;

FIG. 13 is a high level flowchart illustrating a method for manufacturing a paddle electrode assembly stitched onto a sheet substrate in accordance with some embodiments of the present invention;

FIG. 14 illustrates a method of forming a composite yarn in accordance with one embodiment of the fourth and fifth aspects of the invention.

FIG. 15 illustrates a three dimensional rotary braiding machine suitable for braiding a braided fabric paddle electrode assembly in accordance with the first aspect of the invention, using composite filaments in accordance with the fourth aspect of the invention.

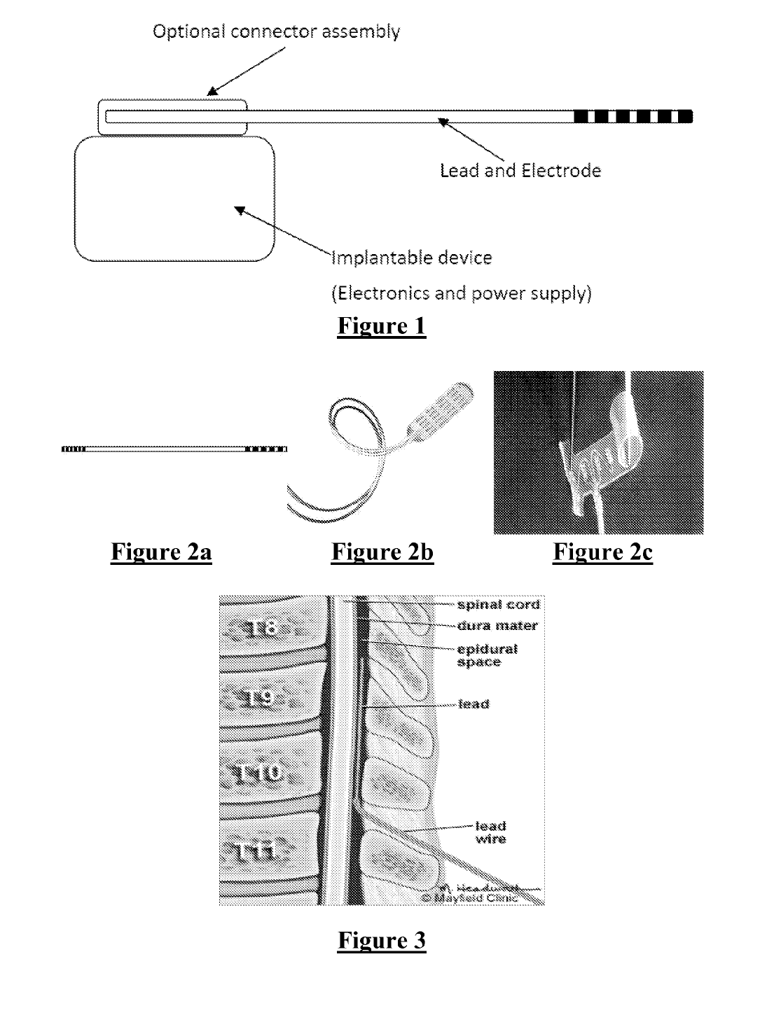

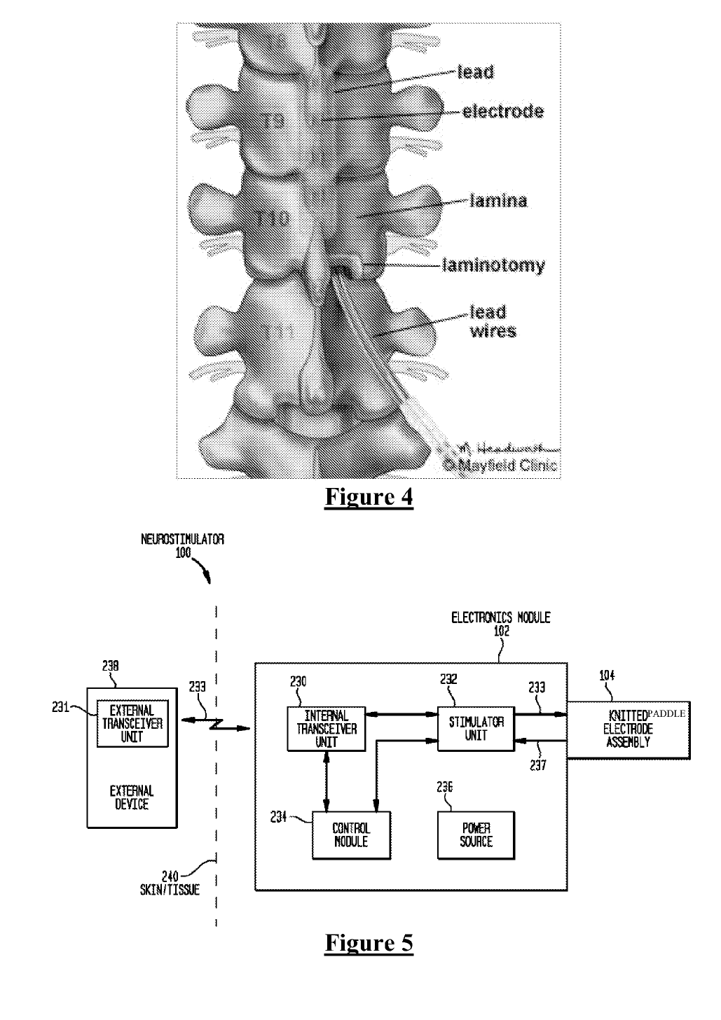

### DESCRIPTION OF THE PREFERRED EMBODIMENTS

FIG. 5 is a system schematic of an active implantable medical device having a knitted paddle electrode assembly in accordance with one embodiment of the invention. Electronics module 102 is implanted under a patient's skin/tissue 240, and cooperates with an external device 238. External device 238 comprises an external transceiver unit 231 that forms a bi-directional transcutaneous communication link 233 with an internal transceiver unit 230 of electronics module 102. Transcutaneous communication link 233 may be used by external device 238 to transmit power and/or data to electronics module 102. Similarly, transcutaneous communication link 233 may be used by electronics module 102 to transmit data to external device 238.

As used herein, transceiver units 230 and 231 each include a collection of one or more components configured to receive and/or transfer power and/or data. Transceiver units 230 and 231 may each comprise, for example, a coil for a magnetic inductive arrangement, a capacitive plate, or any other suitable arrangement. As such, in embodiments of the present invention, various types of transcutaneous communication, such as infrared (IR), electromagnetic, capacitive and inductive transfer, may be used to transfer the power and/or data between external device 238 and electronics module 102.

In the specific embodiment of FIG. 5, electronics module 102 further includes a stimulator unit 232 that generates electrical stimulation signals 233. Electrical stimulation signals 233 are delivered to a patient's tissue via electrodes of the knitted paddle electrode assembly 104. Stimulator unit 232 may generate electrical stimulation signals 233 based on, for example, data received from external device 238, signals received from a control module 234, in a pre-determined or pre-programmed pattern, etc.

As noted above, in certain embodiments, electrodes of the knitted paddle electrode assembly 104 are configured to record or monitor the physiological response of a patient's tissue. In such embodiments, signals 237 representing the recorded response may be provided to stimulator unit 232 for forwarding to control module 234, or to external device 238 via transcutaneous communication link 233.

In the embodiment of FIG. 5, neurostimulator 100 is a totally implantable medical device that is capable of operating, at least for a period of time, without the need for external device 238. Therefore, electronics module 102 further comprises a rechargeable power source 236 that stores power received from external device 238. The power source may comprise, for example, a rechargeable battery. During operation of neurostimulator 100, the power stored by the power source is distributed to the various other components of electronics module 102 as needed. For ease of illustration, electrical connections between power source 236 and the other components of electronics module 102 have been omitted. FIG. 5 illustrates power source 236 located in electronics module 102, but in other embodiments the power source may be disposed in a separate implanted location.

FIG. 5 illustrates specific embodiments of the present invention in which neurostimulator 100 cooperates with an external device 238. It should be appreciated that in alternative embodiments, neural stimulation may be configured to operate entirely without the assistance of an external device.

FIG. 6 illustrates a paddle electrode assembly 600, comprising a supporting member 602 and resiliently deformable paddle wings 612, 614 in accordance with one embodiment of the invention. When wings 612 and 614 are unfurled as shown in FIG. 6a, the paddle wings reside in a common nominal plane which passes through an axis of the supporting member 602. Electrodes (not shown) are formed on each wing, and optionally on the supporting member 602, to form a paddle electrode assembly. When wings 612 and 614 are furled as shown in FIG. 6b, the paddle wings 612, 614 are brought close to and lie against the supporting member 602 so that the electrode assembly 600 presents a smaller cross section so as to fit within a Tuohy needle or the like. In other embodiments the paddle wings 612 and 614 may be wider laterally, so that when furled the distal edge of each wing wraps about halfway around supporting member 602, as far as the base of the other paddle wing. Wings 612, 614 are furled in the same rotational direction, namely clockwise in the view of FIG. 6b.

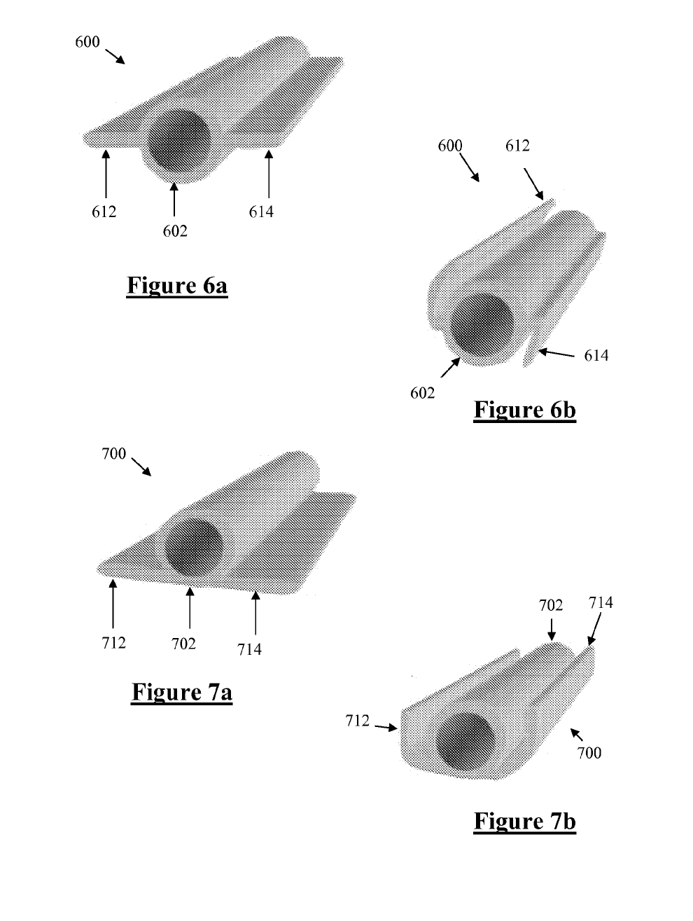

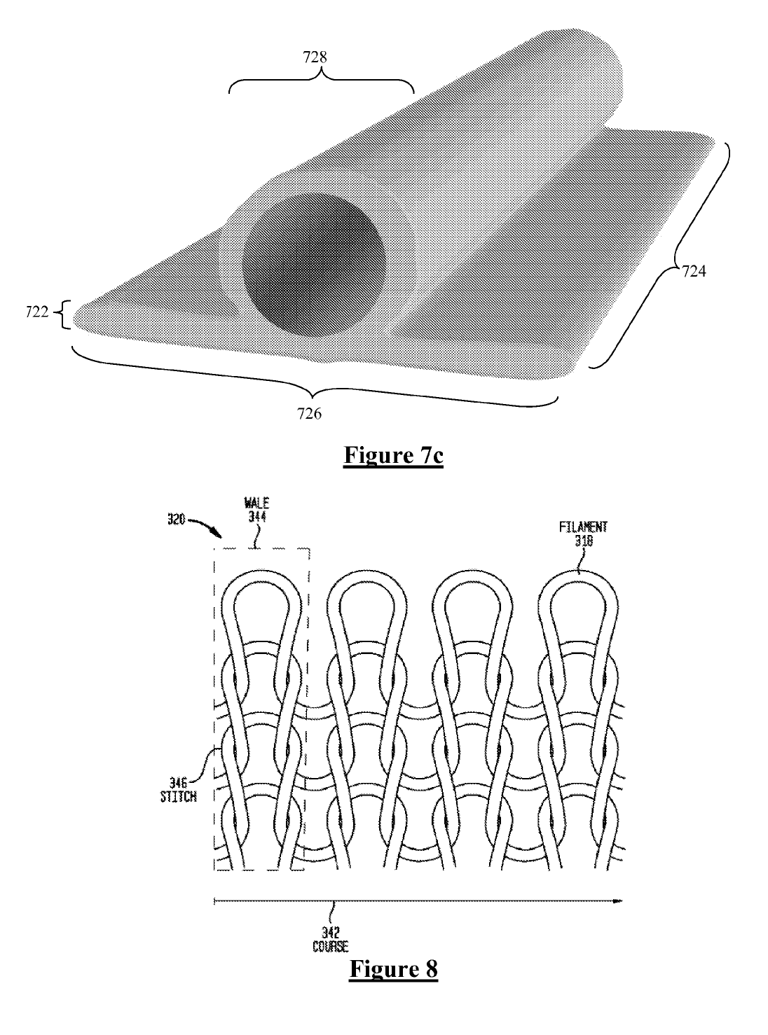

FIG. 7 illustrates a paddle electrode assembly 700 having resiliently deformable paddle wings 712, 714 in accordance with another embodiment of the invention. When wings 712 and 714 are unfurled as shown in FIG. 7a, the paddle wings reside in a common nominal plane which passes tangentially to a cross-sectional circumference of the supporting member 702. Electrodes (not shown) are formed on each wing, and optionally on the supporting member 702, to form a paddle electrode assembly. When wings 712 and 714 are furled as shown in FIG. 7b, the paddle wings 712, 714 are brought close to and lie against the supporting member 702 so that the electrode assembly 700 presents a smaller cross section so as to fit within a Tuohy needle or the like. In other embodiments the paddle wings 712 and 714 may be wider laterally, so that when furled the distal edges of the two wings wrap about halfway around supporting member 702 to be proximal to each other.

FIG. 7c illustrates variable physical parameters of the paddle electrode assembly in accordance with various embodiments of the invention. The paddle thickness 722 may comprise a single layer of stitches or multiple layers of stitches, or a single layer or multilayer substrate, and could for example be in the range of 100-2000 μm. The paddle length 724 is typically about 40-60 mm to cater for 4-8 rows of electrodes. The total paddle width 726 may be 5-15 mm to cater for 2-4 columns of electrodes. The support member may have a substantially circular cross section or other cross section. A diameter 728 of the support member may be in the range of 800-1200 μm. In the preferred embodiment the support member is hollow to allow for the introduction of a removable stylet to stiffen the assembly during placement. Alternatively a shaft may be provided within the support member to provide desired resilience to the electrode assembly and/or to carry conductive wires to the electrodes. In embodiments comprising a fabric structure, the fabric may be knitted or braided onto the shaft so as to be disposed on the surface of the shaft, so that the shaft provides additional mechanical strength to the electrode assembly. The paddle wings may adjoin the support member tangentially as shown in FIG. 7, perpendicularly as shown in FIG. 6, or may adjoin the support member in any suitable alternative fashion.

Still further variations may be made to the deformable paddle electrode array of the present invention. For example a join between the paddle wings and the support member may be perforated to give rise to greater deformation at the join than in the wings themselves when furled. The paddle wings may have constant resilience across their lateral extent or may have variable resilience and/or may be configured to relax to a non-flat and non-circular curve. More than one pair of paddle wings may be provided along the length of the support member.

While FIGS. 6 and 7 show an electrode assembly having paddle wings formed from a sheet substrate, some embodiments of the invention may instead comprise a knitted paddle electrode assembly. A knitted electrode assembly has an inherent ability to change diameter as it is compressed or expanded, in contrast to braided or woven fabric. This allows support structures of various shapes and diameters to be easily introduced, for example.

Such a knitted paddle electrode assembly may in some embodiments comprise at least one biocompatible, electrically non-conductive filament arranged in substantially parallel rows stitched to an adjacent row, with at least one biocompatible, electrically conductive filament intertwined with the non-conductive filament. Knitting is a technique for producing a two or three-dimensional structure from a linear or one-dimensional yarn, thread or other filament (collectively and generally referred to as “filaments” herein). There are two primary varieties of knitting, known as weft knitting and warp knitting. FIG. 8 illustrates a section of a knitted structure 320 formed by weft knitting a single filament 318.

As shown in FIG. 8, the generally meandering path of the filament, referred to as the filament course 342 or as a row of stitches, is substantially perpendicular to the sequences of interlocking stitches 346. This creates substantially straight and parallel rows of filament loops. A sequence of stitches 346 is referred to as a wale 344. In weft knitting, the entire knitted structure may be manufactured from a single filament by adding stitches 346 to each wale 344 in turn. In contrast to the embodiments illustrated in FIG. 8, in warp knitting, the wales run roughly parallel to the filament course 342.

It should be appreciated that embodiments of the present invention may be implemented using weft or warp knitting. Furthermore, embodiments of the present invention may use circular knitting or flat knitting. Circular knitting creates a seamless tube, while flat knitting creates a substantially planar sheet.

Importantly, to effect a composite yarn and thereby form electrodes at desired locations of the electrode assembly, at appropriate moments as the yarn is drawn into the fabric being knitted a conductive filament is wound onto the non-conductive filament to form a conductive portion. Notably, single yarn knitting is important to simply effect this approach as the alternative multi-yarn techniques such as braiding prevent or hamper the ability to wind the conductive filament onto the non-conductive filament.

Electrode assemblies in accordance with embodiments of the present invention may be knitted using automated knitting methods known in the art, or alternatively using a hand knitting process. It should be appreciated that the knitting method, filament diameter, number of needles and/or the knitting needle size may all affect the size of the stitches and the size of the resulting electrode assembly. As such, the size and shape of the assembly is highly customizable.

FIG. 9 illustrates an embodiment of the present invention in which a paddle electrode assembly is formed by alternately knitting with conductive and non-conductive filaments. A portion 420 of a flat paddle portion of such a knitted structure is shown in FIG. 9.

As shown in FIG. 9, a first non-conductive filament 418A is knitted into a plurality of substantially parallel rows 436. A first conductive filament 412 is stitched to one of the rows 436 such that conductive filament 412 forms an additional row 434 that is parallel to rows 436. A second non-conductive filament 418B is stitched to row 434 such that the second non-conductive filament forms one or more rows 432 that are parallel to rows 434 and 436. For ease of illustration, a single conductive row 434 and a single non-conductive row 432 are shown. It should be appreciated that additional conductive or non-conductive rows may be provided in alternative embodiments. It should also be appreciated that in alternative embodiments each conductive row does not necessarily form a full row. For instance, a conductive filament could be used to form a number of stitches within a row or even part of a stitch, and a non-conductive filament could be used to complete the row.

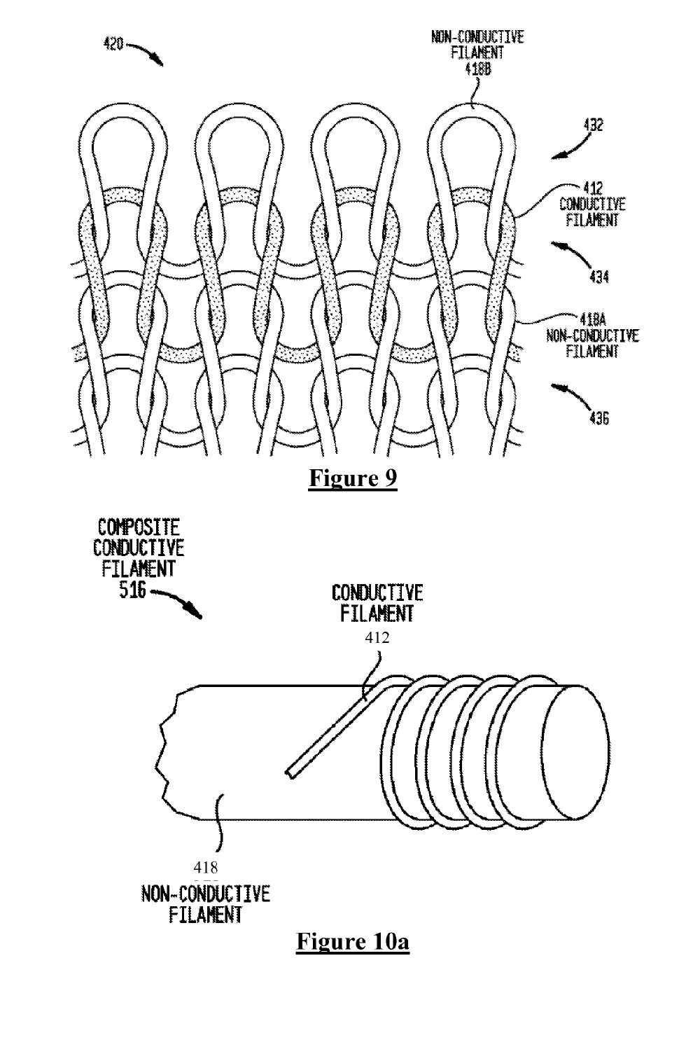

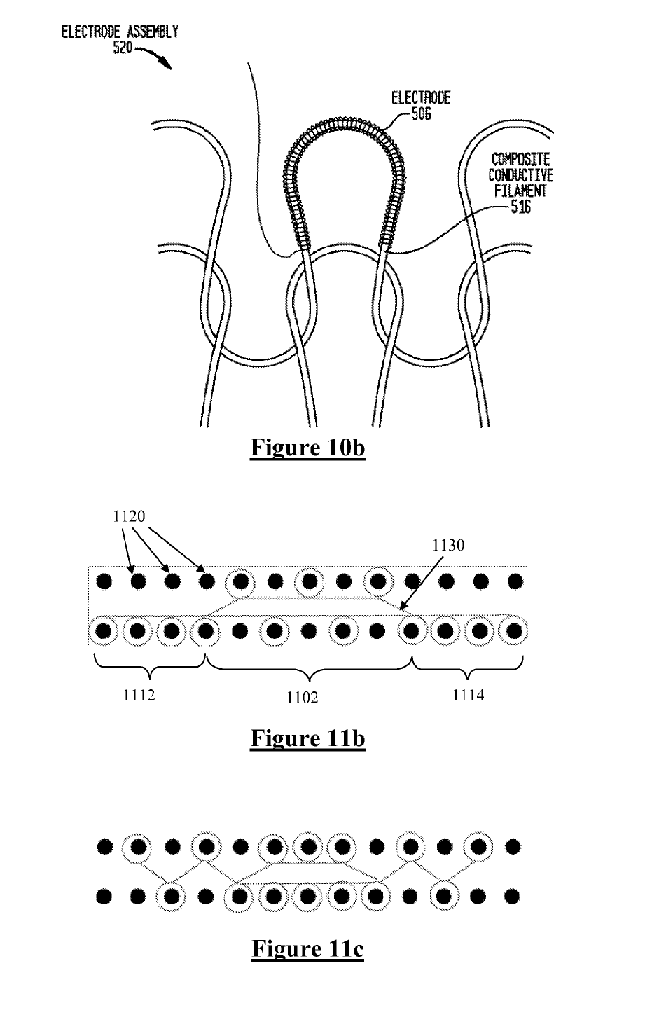

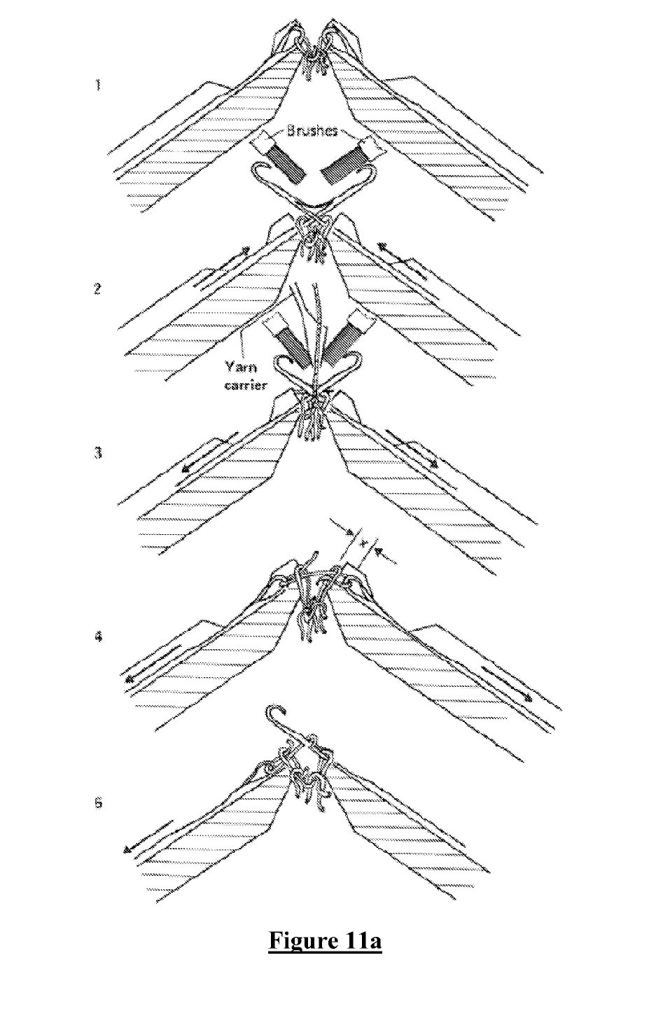

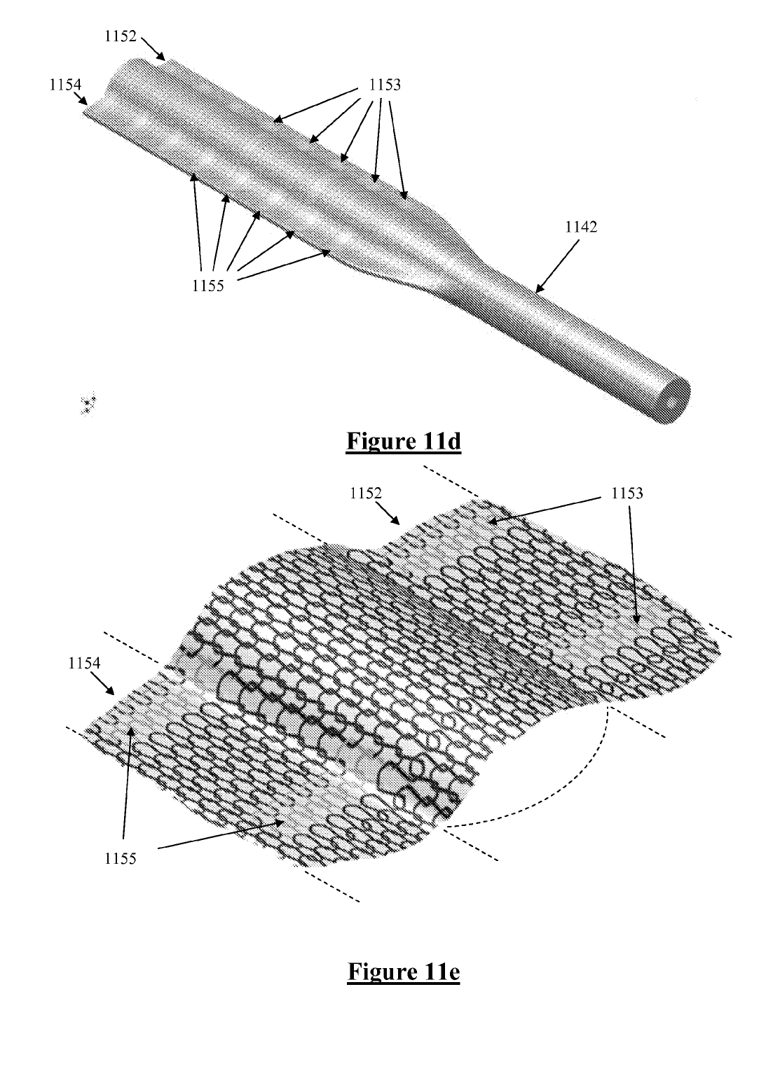

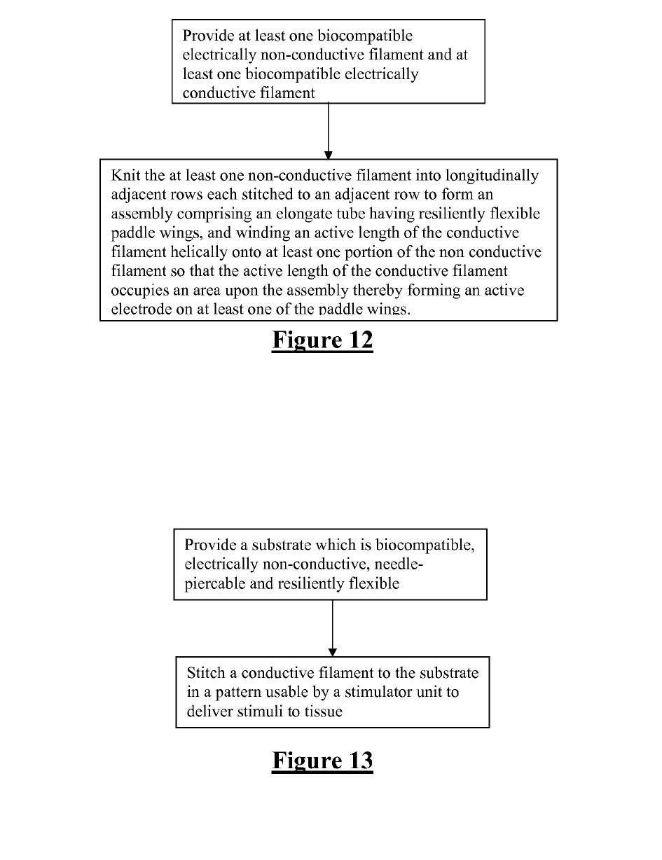

In the specific embodiments of FIG. 9, conductive filaments 412 are conductive threads, fibers, wires or other types of filament that are wound in helical coils around sections of non-conductive filament 418 prior to or during the knitting process. Also as detailed below, the term composite conductive filament is used herein to refer to a non-conductive filament having a conductive filament wound around a section thereof, as shown in FIG. 10a. The conductive filaments 412 may be intertwined with non-conductive filament 418 in one of several other manners. The term “wound” is used herein to refer to wrap or encircle once or repeatedly around a filament. Conductive filament 512 may be loosely or tightly wound onto non-conductive filament 518, and is also referred to herein as being intertwined with non-conductive filament 518.

As noted above, some embodiments of the knitted electrode assembly comprise at least one biocompatible, electrically non-conductive filament arranged in substantially parallel rows stitched to an adjacent row, with at least one biocompatible, electrically conductive filament intertwined with the non-conductive filament. Knitting is a technique for producing a two or three-dimensional structure from a linear or one-dimensional yarn, thread or other filament (collectively and generally referred to as “filaments” herein) to produce an intermeshed loop structure. A stitch in knitting includes the use of one or more loops to connect filaments to form the structure. There are two primary varieties of knitting, known as weft knitting and warp knitting. FIG. 10b illustrates a section of a knitted structure 520 formed by weft knitting a single composite filament 516.

A variety of different types and shapes of conductive filaments may be used to knit an electrode assembly in accordance with embodiments of the present invention. In one embodiment, the conductive filament is a fiber manufactured from carbon nanotubes. Alternatively, the conductive filament is a platinum or other biocompatible conductive wire. Such wires may be given suitable surface treatments to increase their surface area (e.g. forming a layer of iridium oxide on the surface of platinum, utilizing platinum “blacking”, or coating the wire with carbon nanotubes). In other embodiments, the conductive filament comprises several grouped strands of a conductive material. In other embodiments, the filament may be a composite filament formed from two or more materials to provide a desired structure. In certain such embodiments, the properties of the composite filament may change along the length thereof. For example, certain portions of the composite filament may be conductive, while portions are non-conductive. It would also be appreciated that other types of conductive filaments may also be used. Furthermore, although embodiments of the present invention are described using tubular or round fibers, it would be appreciated that other shapes are within the scope of the present invention.

As noted above, conductive filaments in accordance with some embodiments of the present invention are intertwined with a non-conductive filament to form the electrode assembly. While a majority of the intertwined portion is an exposed conductive element, the remainder of the conductive filament may be insulated. In one such embodiment, a length of suitably insulated conductive filament (e.g. parylene coated platinum wire) is provided and the insulation is removed from the section that is to be intertwined, leaving the remainder of the filament with the insulated coating.

A variety of non-conductive filaments may be used to knit an electrode assembly in accordance with embodiments of the present invention. In one embodiment, the non-conductive filament is a biocompatible non-elastomeric polymer material. In another embodiment, the non-conductive filament is a biocompatible elastomeric material. For example, the elastomeric material may comprise, for example, silicone, silicone/polyurethane, silicone polymers, or other suitable materials including AORTech® and PBAX. Other elastomeric polymers that provide for material elongation while providing structural strength and abrasion resistance so as to facilitate knitting, while also providing for resilient deformation of the paddle wings, may also be used. It should be appreciated that other types of non-conductive filaments may also be used.

As noted, the term filament is used to refer to both the conductive and non-conductive threads, fibers or wires that are used to form a knitted electrode assembly. It should be appreciated that, as shown in FIGS. 5A-5C, filaments of varying diameters and properties may be used. As such, the use of filament to refer to both conductive and non-conductive threads, fibers and wires should not be construed to imply that the conductive and non-conductive elements have the same diameter or properties.

In certain embodiments of FIG. 10A, non-conductive filament 418 comprises a thermo-softening plastic material. The use of a thermo-softening filament allows conductive filament 412 to be wound around non-conductive filament 418 while the non-conductive filament is in a softened state. This ensures that conductive filament 412 is well integrated into non-conductive filament 418 so as to reduce any difference in the size of the stitches in the electrode area when compare to those in the non-conductive areas of a formed electrode assembly. As noted, conductive filament 412 may be loosely or tightly wound onto non-conductive filament 418. A loose winding provides integration of the two filaments and provides a compliant structure to manage fatigue. A tight winding provides substantially the same benefits, but also increases the amount of conductive filament in a single stitch.

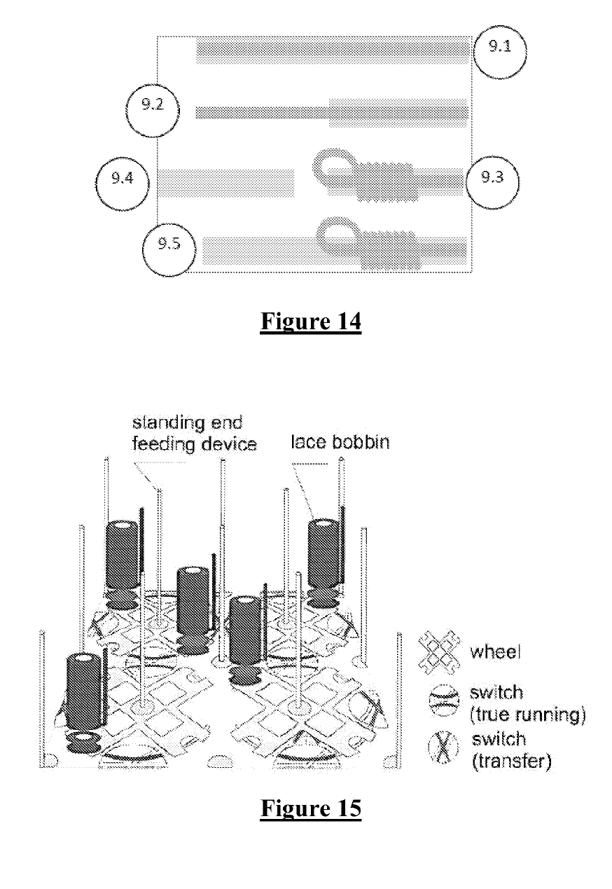

An alternative composite conductive filament is formed using a method as described below with reference to FIG. 14.

When electrode assembly 520 of FIG. 10B is formed, the conductive portions of composite conductive filament 516 (i.e. the portions of conductive filament 412 wound around non-conductive filament 418) form electrode 506 that may be used to deliver electrical stimulation signals to, and/or receive signals from, a patient's tissue. Conductive filament 516 is configured to be electrically connected to an electronics module 102. Thus a section of the filament 516 extends proximally from the intertwined portions of the electrode 506 through the interior of electrode assembly 104 for connection to the electronics module 102.

To fabricate a fabric electrode of the profile shown in FIG. 6 or 7, the present embodiments use 3 dimensional textile techniques. For the purpose of illustration the fabrication of a paddle style electrode which may be introduced percutaneously will be described. It will be understood by those skilled in the art that the textile approach described here can be used to make other lead types, such that these lead types may be introduced using simpler procedures than is currently the state of the art.

For the purposes of illustration this device will be described using a 3 dimensional braiding as the underlying textile technology used to fabricate the device. It will be understood by those skilled in the art that, with small variations to the method, any other 3 dimensional fabric construction method could be used to fabricate the required structure. By using 3 dimensional textile techniques the performance advantages of a paddle electrode may be combined with the minimally invasive placement procedure of the catheter electrode. The present embodiment thus exploits the capacity of these textile techniques to create complex and resilient 3 dimensional structures.

In the examples of FIG. 11 a three dimensional knitted structure corresponding to the device shown in FIG. 7 is constructed using a V-bed weft knitting machine (FIG. 11a). In FIG. 11b the stitch pattern conducted by a plurality of needles 1120 is shown, which creates a knitted fabric electrode assembly having a support member 1102, and paddle wings 1112 and 1114 constructed from yarn 1130. The stitching process is controlled to produce resiliently deformable paddle wings 1112 and 1114. These wings 1112 and 1114 may be furled around the tubular body 1102 of the electrode for placement through an introducer and the resilience in the structure causes the wings to unfurl once they leave the introducer and enter the epidural space. FIG. 11 c illustrates a suitable stitch pattern in accordance with another embodiment of the invention when using a V-bed weft knitting machine.

FIG. 11d illustrates a knitted fabric electrode assembly constructed from a yarn of composite conductive filament and having a support member 1142, and resiliently flexible paddle wings 1152 and 1154. Wing 1152 includes 8 electrode regions 1153, while wing 1154 includes eight electrode regions 1155. The assembly of FIG. 11d is formed in accordance with the knitting technique of FIG. 11a and following the principles of FIG. 11b but having a larger number of wales than the particular configuration shown in FIG. 11b.

FIG. 11e is a detail view of a portion of the knitted fabric electrode assembly of FIG. 11d. Dashed lines in FIG. 11e indicate other portions of the assembly which are not shown in full. As visible in more detail in FIG. 11e, the assembly is formed from a knitted composite conductive filament. Conductive portions of the composite conductive filament form the electrodes 1153 and 1155 which may be used to deliver electrical stimulation signals to, and/or receive signals from, a patient's tissue.

As an alternative to knitting, a fabric paddle electrode in accordance with other embodiments of the present invention can be made by various 3 dimensional fabric methods.

As well as creating the basic electrode assembly structure in the manner shown in FIG. 11, it is also necessary to create conductive elements in the structure to serve as the tissue interface, as discussed elsewhere herein. It is necessary, however, when employing such methods to ensure that the part of the conductive filament that is used to connect the actual tissue interface (at the distal end of the lead) to a connector or AIMD (at the proximal end of the lead) is appropriately managed during the textile creation process.

It is to be understood that some 3D textile techniques, such as weft knitting, are essentially single yarn techniques. In this case it is generally necessary to have a single non-conductive filament to form the basic electrode structure and then one or more conductive filaments to create the electrode elements. Other 3D textile techniques (such as 3-D braiding or warp knitting) require multiple non-conductive filaments for the basic structure and one or more conductive filaments for the electrode elements. In this latter case the management of multiple yarns may become problematic. To address this issue, and as another aspect of the current invention, a method of forming a composite filament with a conductive and non-conductive portion is described. With reference to FIG. 14 the method can be described as follows.

First, a suitable single or multi-strand insulated electrically conductive cable is selected such that the materials are all bio-compatible. (9.1). A suitable length of the insulation is removed from the cable exposing the conductive material with the insulating layer (9.2). The conductive filament(s) in the cable are formed into a helix around an adjacent insulated portion of the cable (9.3), forming an electrode element. A suitable non-conductive filament, preferably made of the same material as the insulating component of the insulated electrically conductive cable and of the same diameter of that cable is brought adjacent to the electrode element formed on the insulated electrically conductive cable (9.4). The non-conductive element is joined to the insulated electrically conductive cable such that it is co-axial with that cable (9.5).

For structures formed from multi-yarn 3D textile techniques one or more “electrode yarns” (or composite filaments) formed by the method described above may be used in the 3-D textile formation method in place of normal insulating yarns. Commonly one “electrode yarn” or composite filament will be used for each separate electrode element in the structure. Each electrode yarn will be arranged in the yarn supply spools so that the conductive element is taken into the textile structure at the appropriate place in the textile structure.

There are many ways of bonding the non-conductive filament with the insulated electrically conductive cable. In the preferred embodiment the insulating materials will be of a thermo-softening character and the materials will be bonded by applying heat at the interface so that the material in the non-conductive filament fuses with the insulation material used in the insulated electrically conductive cable. Other methods such as gluing and knotting are also possible.

Another approach to forming a composite filament or “electrode yarn” is to take a suitable length of nonconductive yarn (which is normally the same or similar to the yarn used in the underlying 3D textile structure) and a suitable length of insulated conductive filament. In the first step a tight helix of stripped wire cable is formed at one end of the yarn, then an open helix of the insulated part of the conductive filament is wound along most to the remaining length of the yarn. This “electrode yarn” can then be introduced into the 3D fabric structure as it is being assembled such that the stripped helix is positioned in the correct part of the 3D structure and then carries the conductive filament through the structure to the connector end of the lead. It will be understood that the conductive filament at the connector end may be handled in various ways to create or connect to a connector assembly.

A composite filament produced in the manner shown in FIG. 14 may then advantageously be used in a 3D rotary braiding method as illustrated in FIG. 15 to fabricate a paddle electrode structure generally of the type shown in FIG. 5.

As noted above, an electrode assembly in accordance with embodiments of the present invention comprises one or more electrodes to deliver electrical stimulation signals to, and/or receive signals from, a patient's tissue. Electrode assemblies in accordance with certain aspects of the present invention may also include one or more other active components configured to perform a variety of functions. As used herein, an active component refers to any component that utilizes, or operates with, electrical signals.

As noted above, the above described knitting methods permit the formation of electrode assemblies having various shapes and sizes. In alternative embodiments of the present invention, a knitted electrode assembly is formed into a desired shape following the knitting process. For example an electrode assembly may be knitted in one of the manners described above from a thermo-softening plastic non-conductive filament, and conductive filament(s). Following the knitting process, the electrode assembly may be placed in a molding apparatus and heat may be applied. Due to the use of a thermosoftening plastic non-conductive filament, the applied heat causes the electrode assembly to take a desired shape.

In one embodiment of the present invention, an electrode assembly may include one or more memory metal filaments, such as Nitinol, knitted into the assembly using one of the methods described above. In such embodiments, the memory metal filaments are preformed to hold the electrode assembly in a first shape prior to implantation in a patient, but is configured to cause the electrode assembly to assume a second shape during or following implantation. The memory metal filaments may also be insulated as required.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. An electrode assembly for an active implantable medical device, the electrode assembly comprising:
a support member carrying wires for electrically connecting a control unit to electrodes of the electrode assembly; and
at least one resilient deformable paddle wing mounted to the support member, the paddle wing being configured to be furled close to the support member under a deformation force to permit implantation via an introducer, and the paddle wing being configured to resiliently unfurl away from the support member upon release of the deformation force, the paddle wing bearing at least one electrode.

2. The electrode assembly of claim 1, comprising first and second paddle wings, the paddle wings configured to extend in substantially opposed directions from the support member when unfurled.

3. The electrode assembly of claim 2 wherein, when viewed along an axis of the support member, the first paddle wing is configured to be furled clockwise around the support member and the second paddle wing is configured to be furled anti-clockwise around the support member.

4. The electrode assembly of claim 2 wherein, when viewed along an axis of the support member, the first and second paddle wings are both configured to be furled in the same direction (whether clockwise or anti-clockwise) around the support member.

5. The electrode assembly of claim 1 wherein the paddle wings are resilient in a manner such that when unfurled the paddle wings seek to return to a planar position in which the paddle wings both reside in a single nominal plane.

6. The electrode assembly of claim 5 wherein the plane of the paddle wings contains a nominal axis of the supporting member.

7. The electrode assembly of claim 5 wherein the plane of the paddle wings is tangential to a cross-sectional profile of the supporting member.

8. The electrode assembly of claim 1 wherein the paddle wings are resilient in a manner such that when unfurled the wings seek to curve away from the cylindrical supporting member.

9. The electrode assembly of claim 1 wherein the electrode assembly comprises a resilient substrate of sheet material.

10. The electrode assembly of claim 9 wherein electrodes are stitched or embroidered upon the substrate of sheet material.

11. The electrode assembly of claim 1 wherein the electrode assembly is formed as a knitted fabric electrode assembly.

12. A method of constructing an electrode assembly for an active implantable medical device, the method comprising:
forming a support member carrying wires for electrically connecting a control unit to electrodes of the electrode assembly; and
forming at least one resilient deformable paddle wing mounted to the support member, the paddle wing being configured to be furled close to the support member under a deformation force to permit implantation via an introducer, and the paddle wing being configured to resiliently unfurl away from the support member upon release of the deformation force, the paddle wing bearing at least one electrode.

13. The method of claim 12, further comprising forming first and second paddle wings, the paddle wings configured to extend in substantially opposed directions from the support member when unfurled.

14. A method of implanting an electrode assembly for an active implantable medical device, the method comprising:
furling one or more resilient paddle wings of the electrode assembly close to a support member of the electrode assembly;
positioning the furled electrode assembly within an introducer;
delivering an outlet of the introducer to a site of desired implantation; and
ejecting the electrode assembly from the outlet while withdrawing the introducer, to thereby position the electrode assembly at the site of desired implantation and to permit the one or more paddle wings to resiliently unfurl.

15. A biocompatible composite filament comprising:
an insulated conductive cable having a conductor and insulating sheath which are both biocompatible;
an exposed length of the conductor, having a free end and a bound end, the free end of the exposed conductor being wound around the outer surface of the filament to form an electrode element; and
a second portion of filament joined to the insulated conductive cable proximal to the bound end of the exposed conductor and in coaxial alignment with the insulated conductive cable.

16. A method of forming a biocompatible composite filament, the method comprising:
providing an insulated conductive cable having a conductor and insulating sheath which are both biocompatible;
stripping a portion of the insulating sheath, to expose a length of the conductor having a free end and a bound end;
winding the free end of the exposed conductor around the outer surface of the filament to form an electrode element; and
joining a second portion of filament to the insulated conductive cable proximal to the bound end of the exposed conductor and in coaxial alignment with the insulated conductive cable.

17. The electrode assembly of claim 1 wherein the electrode assembly is formed as a braided fabric electrode assembly using a biocompatible composite filament comprising:
an insulated conductive cable having a conductor and insulating sheath which are both biocompatible;
an exposed length of the conductor, having a free end and a bound end, the free end of the exposed conductor being wound around the outer surface of the filament to form an electrode element; and
a second portion of filament joined to the insulated conductive cable proximal to the bound end of the exposed conductor and in coaxial alignment with the insulated conductive cable.
